(19) United States
(12) Patent Application Publication
(10) Pub. No.: US 2007/0299276 A1
(43) Pub. Date: Dec. 27, 2007

(54) Preparation of fluoroalkoxystyrenes

(76) Inventor: Andrew Edward Feiring, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/472,753

(22) Filed: Jun. 22, 2006

(51) Int. Cl. C07C 255/32; C07C 69/76; C07C 43/02

(52) U.S. Cl. 558/410; 568/663; 560/62; 560/63

(57) ABSTRACT

A one-step process for the preparation of fluoroalkoxystyrene by contacting fluorinated olefin with a solution of hydroxystyrene is disclosed.

### FIELD OF THE INVENTION

The invention relates to a process for the preparation of fluoroalkoxystyrenes. The invention provides a one-step synthesis of fluoroalkoxystyrenes by contacting fluorinated olefin with a solution of hydroxystyrene.

### BACKGROUND OF THE INVENTION

Fluorinated aromatic compounds, such as fluoroalkoxystyrenes, have potential utility in a wide variety of industrial applications. For example, these fluorinated aromatic compounds are used as monomers for the preparation of polymers, resins, elastomers, coatings, adhesives, automotive finishes and inks. The previously known chemical synthetic methods for producing fluoroalkoxystyrenes require multiple reaction steps, and are also expensive due to the high cost of the starting materials and extensive product purification required. Moreover, large amounts of unwanted byproducts are generated and about half of the starting materials are thus wasted. Therefore, such methods are economically undesirable and raise significant environmental concerns. For example, the process described in EP355652 requires first the synthesis of fluoroalkoxybenzaldehydes, followed by a Wittig reaction to convert the aldehyde group to the fluoroalkoxystyrene product.

It is desired to have a one step method for preparation of fluoroalkoxystyrenes. It is also desirable to have a method for preparation of fluoroalkoxystyrenes with no unwanted byproducts to alleviate or eliminate environmental concerns. The present invention provides such a method.

### SUMMARY OF THE INVENTION

The present invention comprises a process for the preparation of a fluoroalkystyrene of Formula I:

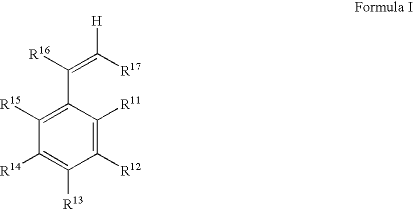

wherein
at least one of groups R¹¹, R¹², R¹³, R¹⁴ and R¹⁵ is —OCF₂CHFYR_{f},
Y is O or a bond;
R_{f} is a straight or branched chain fluoroalkyl group of from 1 to about 20 carbons optionally interrupted with at least one ether oxygen atom, F or Cl;
provided that when Y is O, R_{f} is other than F or Cl;
the remainder of groups R¹¹, R¹², R¹³, R¹⁴ and R¹⁵ are each independently H, F, Cl, Br, I, OH, O(C═O)CH₃, OCH₃, ester, nitrile, linear or branched alkyl, alkyoxyl, fluoroalkyl or fluorinated alkyoxyl chains; and
R¹⁶ and R¹⁷ are each independently H, halo, or cyano;

comprising contacting a hydroxystyrene in at least one polar organic solvent with a fluorinated olefin.

The invention further comprises the product of the above process.

### DETAILED DESCRIPTION OF THE INVENTION

Trademarks are indicated herein by capitalization.

The invention comprises a process for the preparation of fluoroalkoxystyrenes by contacting a fluorinated olefin with a solution of hydroxystyrene. The process results in a higher yield of fluoroalkoxystyrenes than prior art processes with no unwanted byproducts. The process is useful as the resulting fluoroalkoxystyrene compounds have application as monomers for the preparation of polymers, resins, elastomers, coatings, adhesives, automotive finishes and inks.

The following definitions are used herein.

The term “polar” as applied to solvents used in the invention refers to solvents characterized by molecules having sizable permanent dipole moments.

The term “aprotic” as applied to the solvents used in the invention refers to a solvent that is incapable of acting as a labile proton donor or acceptor.

The term “polar organic solvent mixture” refers to a mixture of organic solvents comprising at least one polar solvent.

The term “aprotic, polar organic solvent mixture” refers to a mixture of organic solvents comprising at least one aprotic, polar solvent.

All ranges given herein include the end of the ranges and also all the intermediate range points.

The instant invention comprises a process for the preparation of fluoroalkoxystyrenes, having the general formula I:

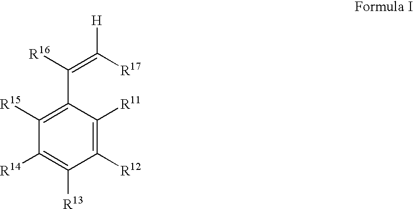

wherein
at least one of groups R¹¹, R¹², R¹³, R¹⁴ and R¹⁵ is —OCF₂CHFYR_{f};
Y is O or a bond;
R_{f} is a straight or branched chain fluoroalkyl group of from 1 to about 20 carbons optionally interrupted with at least one ether oxygen atom, F, or Cl;
provided that when Y is O, R_{f} is other than F or Cl;
the remainder of groups R¹¹, R¹², R¹³, R¹⁴ and R¹⁵ are each independently H, F, Cl, Br, I, OH, O(C═O)CH₃, OCH₃, ester, nitrile, linear or branched alkyl, alkyoxyl, fluoroalkyl or fluorinated alkyoxyl chains; and
R¹⁶ and R¹⁷ are each independently H, halo, or cyano;

comprising contacting a hydroxystyrene in at least one polar organic solvent with a fluorinated olefin.

Examples of suitable R_{f} are CF₃, (CF₂)ₘCF₃ wherein m is 1 to about 20, preferably m is 1 to about 10, and more preferably m is 1 to 2, or [CF₂CF(CF₃)O]ₙ(CF₂)ₚCF₃ wherein n is 1 to 5, and p is 1 to 10. More specific examples of R_{f} include CF₃, CF₂CF₃, CF₂CF₂CF₃, CF(CF₃)₂, CF₂CF(CF₃)OCF₂CF(CF₃)OCF₂CF₂CF₃, and CF₂CF(CF₃)OCF₂CF₂CF₃. More preferably, Y is O and R_{f} is CF₂CF₂CF₃;

An example of the process of the present invention is illustrated by the following equation:

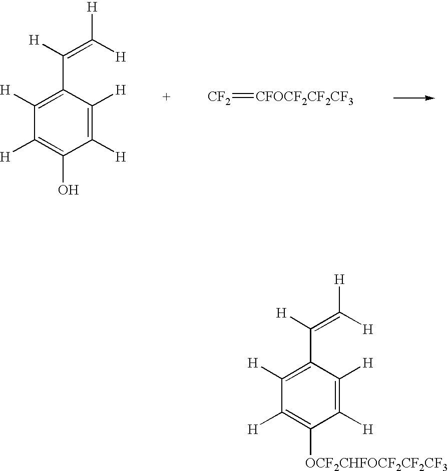

The hydroxystyrenes used as a starting material in the present invention have the general Formula II:

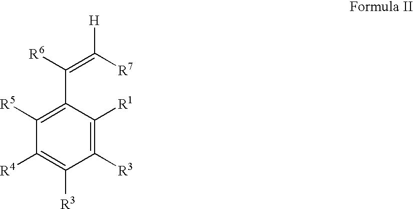

wherein

R¹, R², R³, R⁴ and R⁵ are each independently H, F, Cl, Br, I, O(C═O)CH₃, OH, OCH₃, ester, nitrile, linear or branched alkyl, alkyoxyl, fluoroalkyl or fluorinated alkyoxyl chains;

provided that at least one of R¹, R², R³, R⁴, or R⁵ is OH; and

R⁶, and R⁷ are each independently H, halo, or cyano.

Examples of suitable hydroxystyrenes of Formula II for use in the process of the present invention include 4-hydroxystyrene, 3-methoxy-4-hydroxystyrene, 3,5-dimethoxy-4-hydroxystyrene, 3,4-dihydroxystyrene, 2-hydroxystyrene and α-cyano-4-hydroxystyrene, or a mixture thereof. Preferred for use herein are 4-hydroxystyrene or 3,4-dihydroxystyrene. More preferred are hydroxystyrenes prepared according to US Patent Application 2005/0228191, incorporated herein by reference, which discloses preparing hydroxystyrenes and acetylated derivatives thereof in a solution by thermal decarboxylation of a phenolic substrate in the presence of a non-amine basic catalyst.

The process of the present invention is preferably conducted on the resulting reaction solution in which the hydroxystyrene is produced by the method of U.S. Patent Application 2005/0228191, without isolation of the hydroxystyrene. Examples of the phenolic substrates for use in the method of the U.S. Patent Application 2005/0228191 to produce hydroxystyrenes include 4-hydroxycinnamic acid, ferulic acid, sinapinic acid, caffeic acid, 2-hydroxycinnamic acid, and α-cyano-4-hydroxycinnamic acid. These phenolic substrates are obtained in a number of ways. For example, 4-hydroxycinnamic acid, predominantly in the trans form, is available commercially from companies such as Aldrich (Milwaukee, Wis.) and TCI America (Portland, Oreg.). Additionally, 4-hydroxycinnamic acid is prepared by chemical synthesis using any method known in the art. For example, 4-hydroxycinnamic acid is obtained by reacting malonic acid with para-hydroxybenzaldehyde as described by Pittet et al. in U.S. Pat. No. 4,316,995, or by Alexandratos in U.S. Pat. No. 5,990,336. Alternatively, 4-hydroxycinnamic acid is isolated from plants as described in R. Benrief et al. Phytochemistry 47:825-832 (1998) and U.S. Patent Application 20020187207. In one embodiment, the source of 4-hydroxycinnamic acid is from bioproduction using a production host. In another embodiment, the production host is a recombinant host cell, which may be prepared using standard DNA techniques. These recombinant DNA techniques are described by Sambrook, J., Fritsch, E. F. and Maniatis, T. Molecular Cloning: A Laboratory Manual, Second Edition, Cold Spring Harbor Laboratory Press, Cold Spring Harbor, N.Y. (1989). In another embodiment, 4-hydroxycinnamic acid is produced as described by Qi et al. in U.S. Patent Application 20030079255.

Similarly, ferulic acid, sinapinic acid, and caffeic acid are available commercially from companies such as Aldrich (Milwaukee, Wis.) and TCI America (Portland, Oreg.). Alternatively as these substrates are all natural plant products, comprising elements of the lignin biosynthetic pathway, they are readily isolated from plant tissue (see for example Jang et al., Archives of Pharmacal Research (2003), 26(8), 585-590; Matsufuji et al., Journal of Agricultural and Food Chemistry (2003), 51(10), 3157-3161; WO 2003046163; Couteau et al, Bioresource Technology (1998), 64(1), 17-25; and Bartolome et al., Journal of the Science of Food and Agriculture (1999), 79(3), 435-439). Additionally, methods of chemical synthesis are known for a number of the more common phenolic substrates (see for example WO 2002083625 (“Preparation of ferulic acid dimers and their pharmaceutically acceptable salts, and use thereof for treating dementia&dquor;) JP 2002155017 (“Preparation of caffeic acid from ferulic acids&dquor;); and Taniguchi et al., Anticancer Research (1999), 19(5A), 3757-3761).

The fluorinated olefin suitable as a starting material in the process of the present invention contains a fluoroalkyl or fluoroalkoxy group. The said fluorinated olefin has the general structure of Formula III:

CF₂═CFYR_{f} Formula III

wherein

Y is O or a bond, and

R_{f} is a straight or branched chain fluoroalkyl group of from 1 to about 20 carbons optionally interrupted with at least one ether oxygen atom, F, or Cl;

provided that when Y is O, R_{f} is other than F or Cl.

Examples of suitable R_{f} are CF₃, (CF₂)ₘCF₃ wherein m is 1 to about 20, preferably m is 1 to about 10, and more preferably m is 1 to 2, or [CF₂CF(CF₃)O]ₙ(CF₂)ₚCF₃ wherein n is 1 to 5, and p is 1 to 10. Specific examples of suitable R_{f} include CF₃, CF₂CF₃, CF₂CF₂CF₃, CF(CF₃)₂, CF₂CF(CF₃)OCF₂CF(CF₃)OCF₂CF₂CF₃, and CF₂CF(CF₃)OCF₂CF₂CF₃. More preferably, Y is O and R_{f} is CF₂CFCF₃.

Generally, the fluorinated olefins are prepared as described by Siegemund et. al. in Ullmann's Encyclopedia of Industrial Chemistry, vol. A11, pages 360 to 364 and pages 366 to 367 (1988). The fluorinated olefins which include CF2=CFOCF₂CF₂CF₃, CF2=CFOCF₃ and CF2=CFOCF₂CF(CF₃)OCF₂CF₂CF₃ are commercially available from Synquest Laboratories, Alachua, Fla.

Solvents suitable for use in the process of the present invention include any aprotic, polar organic solvent. A single aprotic, polar solvent is used. Alternatively, mixtures of aprotic, polar solvents, and mixtures of aprotic solvents with nonpolar solvents are used. Aprotic, polar solvents or mixtures thereof are preferred. Examples of suitable aprotic, polar solvents include N,N-dimethylformamide, 1-methyl-2-pyrrolidinone, N,N-dimethylacetamide, dimethylsulfoxide, hexamethylphosphoramide, and hexamethylphosphorous triamide, or a mixture thereof.

About one molar equivalent of fluorinated olefin is required for each mole of fluoroalkoxy group to be introduced into the fluoroalkylstyrene of Formula 1. At least about one mole of fluorinated olefin is used per mole of hydroxy group on the hydroxystyrene to be reacted. In practice, an excess of fluorinated olefin of from 1.01 to 2.0 moles, preferably 1.05 to 1.2 moles, is employed per mole of hydroxy groups to be reacted so as to maximize yield of the desired product.

Polymerization inhibitors are useful but not required in the process of the invention. Any suitable polymerization inhibitor that is tolerant of the temperatures and inert to the conditions required for the reaction is used. Examples of suitable polymerization inhibitors include phenothiazine, N-oxyl(nitroxide) inhibitors, including PROSTAB 5415 which is bis(1-oxyl-2,2,6,6-tetramethylpiperidine-4-yl)sebacate (CAS#25 16-92-9) available from Ciba Specialty Chemicals, Tarrytown, N.Y.; and UVINUL 4040 P which is (1,6-hexamethylene-bis(N-formyl-N-(1-oxyl-2,2,6,6-tetramethylpiperidine-4-yl)amine, available from BASF Corp., Worcester, Mass.

The solution of hydroxystyrene, the organic solvent, and fluorinated olefin are contacted in a reaction vessel to form a reaction mixture. Any suitable reaction vessel is used. If the reaction is to be conducted at a temperature above the boiling point of the fluorinated olefin, use of a pressured vessel is preferred. Typically, the reaction is conducted at temperatures in the range of from about ambient temperature to about 100° C. Preferably, temperatures are in the range of from about 22° C. to about 60° C.

The reaction is conducted in the presence of a catalytic amount of a base with sufficient strength to cause ionization of the phenolic group under the reaction conditions. Typical bases include alkali metal hydroxides, alkali metal alkoxides and alkali metal hydrides. Preferred bases include potassium hydroxide, potassium tert-butoxide and sodium hydride. The base is present in amounts ranging from 0.01 to 100 mole percent of the phenolic groups. Preferred amounts are from about 1 to 20 mole percent.

The reaction is carried out at a pressure ranging from atmospheric pressure to about 1000 psig (6895 kPa), preferably from atmospheric pressure to a pressure of about 500 psig (3447 kPa). Typically, the reaction is conducted at atmospheric pressure. The pressure is adjusted using an inert gas such as nitrogen. For reactions at elevated pressures, any conventional pressure reaction vessel is used including, for example, shaker vessels, rocker vessels, and stirred autoclaves.

There is no limit on the time for the reaction; however, most reactions will run in less than 24 hours and reaction times of about 6 hours to about 12 hours are typical.

The process of the present invention is useful to provide fluoroalkoxystyrene in a single step process. The fluoroalkoxystyrene obtained has a very high level of purity, often greater than 99%. This is a substantial advantage over previously known processes which often provided product containing high levels, sometimes as much as about 50%, of unwanted byproducts. Thus the process of the present invention is economical, and does not raise environmental concerns regarding disposal of unwanted byproducts.

### EXAMPLES

Unless otherwise specified, all chemicals used in the following Examples were reagent grade and were obtained from Sigma-Aldrich (St. Louis, Mo.).

### Example 1

A solution of 30 g of 4-hydroxystyrene and 140 mL of N,N-dimethylacetamide was added with 2.5 g of potassium hydroxide pellets to a 400 mL Hastelloy pressure vessel. The vessel was closed, cool in dry ice acetone, evacuated and charged with 80 g of perfluoropropyl vinyl ether. The vessel was heated to 60° C. and shaken for 8 hr. It was cooled to room temperature and vented to 1 atmosphere. The vessel contents were poured into 600 mL of water and extracted with 2×300 mL of ether. The combined ether extracts were washed with 2×200 mL of water, dried over anhydrous magnesium sulfate and concentrated on a rotary evaporator to 96.2 g of oil. Distillation afforded 72 g of the product, bp 58-60° C. at 0.8 mm. The final product was identified as 4-[2-(heptafluoropropoxy)-1,1,2-trifluoroethoxy]styrene by ¹H-¹⁹F NMR measurement using 400 MHz BRUKER NMR instrument.

### Example 2

A solution of 27.2 g of 3,4-dihydroxystyrene and 150 mL of N,N-dimethylacetamide was added with 2.5 g of potassium hydroxide pellets to a 400 mL Hastelloy pressure vessel. The vessel was closed, cool in dry ice acetone, evacuated and charged with 133 g of perfluoropropyl vinyl ether. The vessel was heated to 60° C. and shaken for 8 hr. It was cooled to room temperature and vented to 1 atmosphere. The vessel contents were poured into 600 mL of water and extracted with 300 mL methylene chloride. The methylene chloride solution was washed with water, dried over anhydrous magnesium sulfate and concentrated on a rotary evaporator to 118 g of oil. Distillation afforded 91 g of the product, bp 85-92° C. at 0.22 mm. The final product was identified as 3,4-bis[2-(heptafluoropropoxy)-1,1,2-trifluoroethoxy]styrene by ¹H-¹⁹F NMR measurement using 400 MHz BRUKER NMR instrument.

### Example 3

A 1-L round bottom flask with dry ice condenser, addition funnel and magnetic stirrer was swept with nitrogen and charged with a solution of 27.2 g (0.2 mol) of 3,4-dihydroxystyrene, 123.8 g of N,N-dimethylacetamide and 2.5 g of potassium hydroxide. Perfluoropropyl vinyl ether (88.6 mL, 133 g, 0.5 mol) was added dropwise from the addition funnel with the solution temperature rising to 44° C. After addition was complete, the solution was allowed to stir for 1.5 hr at room temperature. Methylene chloride (200 mL) was added and the solution was washed with 3×500 mL of water. The organic solution was dried over anhydrous magnesium sulfate and concentrated on a rotary evaporator to 103.4 g of oil. A second reaction was run under the same procedure using twice the quantities of reagents with the reaction temperature held at 25-28° C. during the addition of the perfluoropropyl vinyl ether. This mixture was allowed to stir overnight at room temperature. Isolation using methylene chloride and water as described above provided 208.1 g of crude product. The products were combined and distilled through a short Vigreux column giving 202 g of product, bp 94-96° C. at 0.20-0.25 mm which was found to be 99.7% pure by gas chromatographic analysis. The final product was identified as 3,4-bis[2-(heptafluoropropoxy)-1,1,2-trifluoroethoxy]styrene by ¹H-¹⁹F NMR measurement using 400 MHz BRUKER NMR instrument: ¹H NMR (δ, CDCl₃) 5.32 (d, 1H), 5.72 (d, 1H), 6.04 (d, 2H), 6.62 (dd, 1H), 7.27 (s, 2H), 7.37 (s, 1H); ¹⁹F NMR(δ, CDCl₃) −82.0 85.2 to −88.0 (8F), −130.2 (4F), −144.5 (2F).

What is claimed is:

1. A process for the preparation of a fluoroalkystyrene of Formula I: 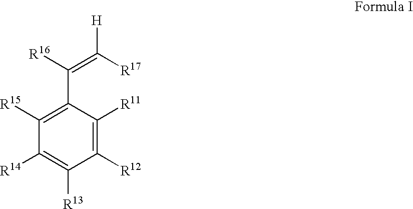
wherein
at least one of groups R¹¹, R¹², R¹³, R¹⁴ and R¹⁵ is —OCF₂CHFYR_{f};
Y is O or a bond;
R_{f} is a straight or branched chain fluoroalkyl group of from 1 to about 20 carbons optionally interrupted with at least one ether oxygen atom, F or Cl;
provided that when Y is O, R_{f} is other than F or Cl;
the remainder of groups R¹¹, R¹², R¹³, R¹⁴ and R¹⁵ are each independently H, F, Cl, Br, I, OH, O(C═O)CH₃, OCH₃, ester, nitrile, linear or branched alkyl, alkyoxyl, fluoroalkyl or fluorinated alkyoxyl chains; and
R¹⁶and R¹⁷ are each independently H, halo, or cyano;
comprising contacting a hydroxystyrene in at least one polar organic solvent with a fluorinated olefin.

2. The process of claim 1 wherein R_{f} is a straight or branched chain fluoroalkyl group of from 1 to about 20 carbons optionally interrupted with at least one ether oxygen atom.

3. The process of claim 2 wherein R_{f} is CF₃, (CF₂)ₘCF₃ wherein m is 1 to about 20, or [CF₂CF(CF₃)O]ₙ(CF₂)ₚCF₃ wherein n is 1 to 5, and p is 1 to 10.

4. The process of claim 3 wherein m is 1 to about 10.

5. The process of claim 3 wherein m is 1 to 2.

6. The process of claim 3 wherein R_{f} is CF₃, CF₂CF₃, CF₂CF₂CF₃, CF(CF₃)₂, CF₂CF(CF₃) OCF₂CF(CF₃)OCF₂CF₂CF₃ or CF₂CF(CF₃)OCF₂CF₂CF₃.

7. The process of claim 6 wherein R_{f} is CF₂ CF₂CF₃.

8. The process of claim 1, wherein Y is O.

9. The process of claim 1 wherein the hydroxystyrene has the general formula II: 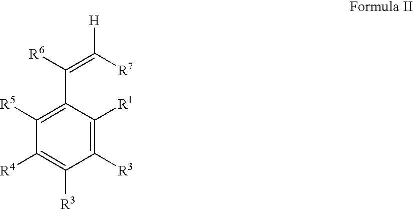
wherein
R¹, R², R³, R⁴ and R⁵ are each independently H, F, Cl, Br, I, OH, O(C═O)CH₃, OCH₃, ester, nitrile, linear or branched alkyl, alkyoxyl, fluoroalkyl or fluorinated alkyoxyl chains;
provided that at least one of R¹, R², R³, R⁴ or R⁵ is OH; and
R⁶ and R⁷ are each independently H, halo, or cyano.

10. The process of claim 9 wherein the hydroxystyrene is 4-hydroxystyrene, 3-methoxy-4-hydroxystyrene, 3,5-dimethoxy-4-hydroxystyrene, 3,4-dihydroxystyrene, 2-hydroxystyrene or α-cyano-4-hydroxystyrene, or a mixture thereof.

11. The process of claim 1 wherein the fluorinated olefin contains a fluoroalkyl or fluoroalkoxy group.

12. The process of claim 1 wherein the fluorinated olefin is:
CF₂═CFYR_{f} Formula III
wherein
Y is O or a bond, and
R_{f} is a straight or branched chain fluoroalkyl group of from 1 to about 20 carbons optionally interrupted with at least one ether oxygen atom, F or Cl;
provided that when Y is O, R_{f} is other than F or Cl.

13. The process of claim 12 wherein R_{f} is CF₃, CF₂CF₃, CF₂CF₂CF₃, CF(CF₃)₂, CF₂CF(CF₃)OCF₂CF(CF₃)OCF₂CF₂CF₃ or CF₂CF(CF₃)OCF₂CF₂CF₃.

14. The process of claim 1 wherein the solvent comprises N,N-dimethylformamide, 1-methyl-2-pyrrolidinone, N,N-dimethylacetamide, dimethylsulfoxide, hexamethylphosphoramide, hexamethylphosphorous triamide, or a mixture thereof.

15. The process of claim 1 comprising contacting the hydroxystyrene with the fluorinated olefin at a temperature of from ambient temperature to about 100° C.

16. The process of claim 15 comprising contacting the hydroxystyrene with the fluorinated olefin at a temperature of from about 22° C. to about 60° C.

17. The process of claim 1 wherein at least about one mole of fluorinated olefin is present per mole of hydroxy group on the hydroxystyrene.

18. The product of the process of claim 1.
